(19) United States
(12) Patent Application Publication
(10) Pub. No.: US 2024/0050388 A1
(43) Pub. Date: Feb. 15, 2024

(54) CALCIUM SUCCINATE MONOHYDRATE FORMULATION

(71) Applicant: BioLink Life Sciences, Inc., Cary, NC (US)

(72) Inventor: Deanna J. Nelson, Raleigh, NC (US)

(21) Appl. No.: 18/233,398

(22) Filed: Aug. 14, 2023

Related U.S. Application Data
(60) Provisional application No. 63/398,084, filed on Aug. 15, 2022.

(51) Int. Cl. A61K 31/194; A61K 47/38; A61K 9/16

(52) U.S. Cl. CPC A61K 31/194; A61K 47/38; A61K 9/1641

(57) ABSTRACT

This invention relates to a ready to use, orally administered formulation containing a therapeutically effective amount of calcium as calcium succinate monohydrate with unexpected palatability, chemical and physical stability, and availability for phosphate binding in the gastrointestinal tract of a subject.

### CROSS-REFERENCE TO RELATED APPLICATIONS

This application claims the benefit of U.S. Provisional Application No. 63/398,084, filed Aug. 15, 2022, the content of which is herein incorporated by reference in its entirety.

### FIELD OF THE INVENTION

This invention relates to a ready to use, orally administered formulation containing a therapeutically effective amount of calcium as calcium succinate monohydrate with unexpected palatability, chemical and physical stability, and availability for phosphate binding in the gastrointestinal tract of a subject.

### BACKGROUND OF THE INVENTION

Hyperphosphatemia is a condition in which there is an abnormally elevated level of phosphate in the serum. It develops in the majority of patients with end-stage renal disease (ESRD) and is associated with secondary hyperparathyroidism, metabolic bone disease, soft tissue calcification, and increased mortality. Therefore, adequate control of serum phosphate remains a cornerstone in the clinical management of patients with ESRD. Management is achieved by dietary phosphate restriction, and through dialysis and the use of oral phosphate binders. Since restricting phosphate in the diet and phosphate removal by conventional dialysis regimens provide limited benefit, the use of phosphate binders constitutes a major therapeutic approach to maintaining phosphate balance and preventing hyperphosphatemia. Clinically used phosphate binders include calcium salts, such as calcium carbonate and calcium acetate; resins such as sevelamer chloride and carbonate; lanthanum carbonate; and iron preparations such as sucroferric oxyhydroxide and ferric citrate.

Since its introduction in the late 1990's, calcium acetate has exhibited both safe and effective phosphate binding at reasonable cost to the hyperphosphatemic subject. When given orally, calcium acetate dissolves in gastric fluid and ionizes, releasing calcium ion. At pH 1, the nominal pH of an empty stomach, both calcium and phosphate ions are soluble. However, as the pH increases to pH 4-6 following ingestion of food, calcium phosphates precipitate. With further increases in pH to 6.8 and higher in the intestinal tract, phosphate-binding by the calcium ion goes to near-quantitative completion. The calcium phosphates that form are insoluble and are not absorbed from the gastrointestinal tract but are excreted in the feces. In vivo phosphate binding by calcium acetate is essentially quantitative.

Calcium acetate offers the advantage that it is highly soluble in acidic solutions. Thus, it is an effective phosphate binder when taken before or with food. However, when calcium acetate is acidified, acetic acid is released. Acetic acid is volatile and causes acid reflux; chronic exposure to acetic acid also leads to respiratory irritation. Both acid reflux and respiratory irritation may reduce compliance with dosage regimens.

Calcium succinate is another calcium salt that is soluble at acid pH but one which, after acidification, generates non-volatile succinic acid. Therefore, phosphate-binding by calcium succinate was examined. The inventor/she discovered that phosphate binding by calcium as calcium succinate was equivalent to that of calcium as calcium acetate. Further, she discovered that hydroxypropylmethylcellulose capsules containing formulations of calcium succinate monohydrate could be manufactured at commercial scales using conventional encapsulation equipment in compliance with Current Good Manufacturing Practices to meet rigorous product release and stability specifications. She discovered that after storage at 25° C./40% relative humidity or 40° C./75% relative humidity for a year or longer, the calcium succinate capsules remained stable and did not undergo deterioration or degradation.

However, as additional batches of calcium succinate capsules were manufactured under these conditions, she unexpectedly discovered that the capsules exhibited widely different dissolution profiles at the time of product release, differences which caused the capsules to fail rigorous product specifications for release of the product to commerce. Her research, which is described in detail in the Examples, showed that the root causes for failure to meet dissolution specifications were related to the encapsulation process.

### SUMMARY OF THE INVENTION

The present invention provides a single dose, ready to use formulation containing calcium succinate monohydrate as a unique active ingredient with the aim of obtaining a palatable formulation that exhibited chemical and physical stability and dissolved completely in water or aqueous solutions within 30 minutes (i.e., was an “immediate release” formulation). The stability of this unique solid dosage form has been demonstrated in primary packaging and under different storage conditions, which showed that the product meets rigorous pharmaceutical specifications even under accelerated conditions intended to induce degradation.

Therefore, one embodiment of the invention provides a ready to use calcium succinate monohydrate formulation comprising: (a) calcium succinate monohydrate or a pharmaceutically acceptable anhydrous form thereof; (b) a disintegrant; (c) a binder; and (d) a lubricant.

In another embodiment the invention provides a calcium succinate formulation in a ready to use stick pack comprising: (a) calcium succinate monohydrate or a pharmaceutically acceptable anhydrous form thereof; (b) sodium starch glycolate; (c) silicified microcrystalline cellulose; and (d) sodium dodecyl sulfate.

In yet another embodiment the invention provides a calcium succinate formulation in a ready to use stick pack comprising: (a) calcium succinate monohydrate or a pharmaceutically acceptable anhydrous form thereof; (b) 5.5-6.5% by weight sodium starch glycolate; (c) 1.4-1.6% by weight silicified microcrystalline cellulose; and (d) 0.4-0.6% by weight sodium dodecyl sulfate, wherein the formulation is provided in a ready to use stick pack.

In yet another embodiment the invention provides a method of treating a condition known as hyperphosphatemia in a patient in need thereof comprising administering to said patient a therapeutically effective amount of the ready to use calcium succinate formulation of the present invention, preferably comprising: (a) calcium succinate monohydrate or a pharmaceutically acceptable anhydrous form thereof; (b) sodium starch glycolate; (c) silicified microcrystalline cellulose; and (d) sodium dodecyl sulfate, wherein the formulation is provided in a ready to use stick pack.

In some preferred embodiments, the stick pack comprises a laminate sheet that is in contact with the formulation and wherein the laminate sheet is formed from polyethylene or polypropylene.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that the descriptions embodied in the specification are intended to provide sufficient detail for one skilled in the art to understand the invention and are not intended to limit the invention.

### BRIEF DESCRIPTION OF THE FIGURES

FIGS. 1(a) and 1(b) are graphs of capsule dissolution profiles for (a) manually filled capsules containing calcium succinate (CaS) lot 32557001 and (b) calcium succinate capsules (Drug Product) containing the same lot of calcium succinate that were manufactured at a commercial manufacturing site.

FIGS. 2(a) and 2(b) are graphs of capsule dissolution profiles for (a) manually filled capsules containing calcium succinate (CaS) lot 32550001 and (b) calcium succinate capsules containing the same lot of calcium succinate that were manufactured at a commercial manufacturing site.

FIG. 3 is a graph of the dissolution profiles of calcium succinate capsules (Drug Product) manufactured during a tamping study carried out at the commercial manufacturing site. The numbers 1-6 refer to encapsulation runs completed with variations in the encapsulation equipment settings.

### DEFINITIONS

As used in this specification and in the claims which follow, the singular forms “a”, “an” and “the” include plural referents unless the context clearly dictates otherwise.

“Disintegrants” means compounds that are added to encapsulated formulations to promote the dispersion of the capsule contents into smaller fragments in an aqueous environment, thereby increasing the available surface area and promoting a more rapid release and dissolution of the drug substance. Sodium starch glycolate NF was selected as the preferred disintegrant and constitutes about 6% by weight of the drug product formulation. This concentration is pharmaceutically suitable for its intended purpose.

“Binders” are compounds that impart cohesiveness to a formulation and improve the free-flowing qualities by the formulation of granules of desired hardness and size. Silicified microcrystalline cellulose NF was selected as the preferred binder and constitutes about 1.6% by weight of the drug product formulation. This concentration is pharmaceutically suitable for its intended purpose.

“Lubricants” are compounds that aid in compaction and automated transfer of a formulation to empty capsules. Sodium lauryl sulfate was selected as the preferred lubricant and constitutes about 0.5% by weight of the drug product formulation. This concentration is pharmaceutically suitable for its intended purpose.

“Therapeutically effective amount” means that amount which, when administered to a human for supporting or affecting a metabolic process, or for treating or preventing a disease, is sufficient to cause such treatment or prevention of the disease, or support or affect the metabolic process.

When used herein, the term “about” will correlate with the variability allowed for in the pharmaceutical industry and inherent in this industry, such as differences in product strength due to manufacturing variation and time-induced product degradation. The term allows for any variation which in the practice of Current Good Manufacturing Practices would allow the product being evaluated to be considered therapeutically equivalent or bioequivalent in humans to the recited strength of a claimed product.

The term “Current Good Manufacturing Practices” refers to those actions associated with drug product manufacturing, testing, or storage that are defined by law, the International Committee on Harmonization or the U.S. Food and Drug Administration.

The term “Immediate Release” refers to a Drug Product having an active pharmaceutical ingredient that is released from its dosage form and/or formulation within 30 minutes under defined conditions of dissolution.

When used herein, the term “treatment” means to reduce the occurrence of a symptom or condition, or to relieve or alleviate at least one symptom associated with such condition, or to slow or reverse the progression of such condition, or to manage or affect the metabolic processes underlying such condition. Within the meaning of the present invention, the term also denotes to arrest, delay the onset (i.e., the period prior to clinical manifestation of a disease) and/or reduce the risk of developing or worsening a disease.

A phrase containing the word “acceptable” as used in connection with compositions of the invention, refers to molecular entities and other ingredients of such compositions that are physiologically tolerable and do not typically produce untoward reactions when administered to a subject (e.g., a mammal such as a human).

As used herein, the term “stick pack” refers to a sachet-type package that contains a single dosage of an active agent.

### DETAILED DESCRIPTION OF THE INVENTION

The present invention provides stick packs that contain a therapeutically effective amount of calcium provided calcium succinate monohydrate, also referred to as calcium succinate.

In some preferred embodiments, the therapeutically effective amount of calcium provided as calcium succinate is from 69 to 269 mg of calcium provided as calcium succinate or any range or value therein (e.g., 79 to 259, 89 to 249, 99 to 239, 109 to 229, 119 to 219, 129 to 209, 139 to 199, 149 to 189, 159 to 179 or about 129, 139, 149, 159, 169, 179, 189, 199, 209, 219 or 229 mg of calcium provided as calcium succinate). In some preferred embodiments, the therapeutically effective amount of calcium provided as calcium succinate is about 169 mg calcium provided as calcium succinate. In some preferred embodiments, the therapeutically effective amount of calcium provided as calcium succinate is 169 mg calcium provided as calcium succinate.

The calcium succinate may be formulated in the stick packs as a single, double or triple dosage. In some embodiments, the stick pack contains a single dosage of calcium succinate, i.e., from 69 to 269 mg of calcium provided as calcium succinate or any range or value therein (e.g., 79 to 259, 89 to 249, 99 to 239, 109 to 229, 119 to 219, 129 to 209, 139 to 199, 149 to 189, 159 to 179 or about 129, 139, 149, 159, 169, 179, 189, 199, 209, 219 or 229 mg of calcium provided as calcium succinate). In some embodiments, the stick pack contains a double dosage of calcium succinate, i.e., from 288 to 388 mg of calcium provided as calcium succinate or any range or value therein (e.g., 298 to 378, 308 to 368, 318 to 358, 328 to 348 or about 308, 318, 328, 338, 348, 358, 368 or 378 mg of calcium provided as calcium succinate). In some embodiments, the stick pack contains a triple dosage of calcium succinate, i.e., from 457 to 557 mg of calcium provided as calcium succinate or any range or value therein (e.g., 467 to 547, 477 to 537, 487 to 527, 497 to 517 or about 467, 477, 487, 497, 507, 317, 527, 537 or 547 mg of calcium provided as calcium succinate).

The therapeutically effective amount of calcium provided as calcium succinate may be formulated with a pharmaceutically acceptable agents such as one or more of a disintegrant, binder and lubricant. Suitable agents may be selected from Remington: The Science and Practice of Pharmacy, 21^{st} Ed., Lippincott Williams & Wilkins, Baltimore, Md. (2003). In some preferred embodiments, the formulation releases at least about 80% of the calcium within 30 minutes when dissolved in water at a temperature of 37±0.5° C. that is stirred by paddles rotating at a rate of 50 RPM.

In some preferred embodiments, the formulation comprises from about 5.5 to about 6.5 weight percent of a disintegrant (or any range or value therein, e.g., 5.6 to 6.4, 5.7 to 6.3, 5.8 to 6.2 or 5.9 to 6.1 weight percent). Disintegrants are excipients that aid in dispersion, normally via hydration. Suitable disintegrants include but are not limited to, sodium starch glycolate, starch, crosslinked alginic acid, sodium alginate, cellulose, methylcellulose, crosslinked cellulose, microcrystalline cellulose, crosslinked polyvinylpyrrolidone and soy polysaccharides.

In some preferred embodiments, the formulation comprises from about 1.4 to about 1.6 weight percent of a binder (or any range or value therein, e.g., 1.45 to 1.55, 1.47 to 1.53, or 1.49 to 1.51 weight percent). Binders are excipients that impart enhanced cohesiveness to a pharmaceutical composition. Suitable binders include, but are not limited to, silicified microcrystalline cellulose, starch, pregelatinized starch, poly(ethylene glycol) (PEG), sorbitol, and hydroxypropyl methylcellulose.

In some preferred embodiments, the formulation comprises from about 0.4 to about 0.6 weight percent of a lubricant (or any range or value therein, e.g., 0.45 to 0.55, 0.47 to 0.53, or 0.49 to 0.51 weight percent). Lubricants are agents that aid in reducing friction during the processing of a pharmaceutical agents by a machine such as a filler. Suitable lubricants include, but are not limited to, water soluble lubricants such as sodium lauryl sulfate, boric acid, Carbowax (PEG), and sodium oleate.

The materials used to construct the laminate sheet for the stick pack can be any that are customary in the art, such as polyester, polypropylene, polyethylene, and polyethylene terephthalate (PET), provided that the stick pack is sufficiently tear resistant until correctly manipulated. Because direct contact with mineral salts such as calcium succinate for prolonged periods of time may transfer aluminum from an aluminum surface to the mineral salt, in preferred embodiments the layer of the laminate sheet for the stick pack that is in contact with a formulation of the invention is polyethylene or polypropylene.

It is contemplated that the formulations of the present invention, contained within a stick pack, find use for the treatment of any disease condition where calcium supplementation is indicated. In some preferred embodiments, the formulations are utilized for treating hyperphosphatemia in a patient in need thereof.

### EXAMPLES

In the following Examples, efforts have been made to provide those of ordinary skill in the art with a complete disclosure and description of how the methods claimed herein are made and evaluated and are intended to be purely exemplary of the invention and are not intended to limit the scope of what the inventor regards as her invention.

### Brief Description of the Formulation

The formulation of each unit dose of the Calcium Succinate Capsule Drug Product is shown in Table 1.

TABLE 1
Calcium Succinate Capsule Unit Dose Formulation

| | Weight Percent | Weight Per |
|---|---|---|
| Ingredient | Per Capsule | Capsule (mg) |
| Calcium succinate•H₂O | 91.9 | 735.0 |
| Sodium starch glycolate, NF | 6.0 | 48.0 |
| Silicified microcrystalline cellulose, NF | 1.6 | 13.0 |
| Sodium lauryl sulfate, NF | 0.5 | 4.0 |
| Total | 100.0 | 800.0 |

The formulation of each unit dose of the Calcium Succinate Stick Pack Drug Product is shown in Table 2.

TABLE 2
Calcium Succinate Capsule Unit Dose Formulation

| | | Weight Per |
|---|---|---|
| | Weight Percent | Stick |
| Ingredient | Per Capsule | Pack (mg) |
| Calcium succinate•H₂O | 91.9 | 735.0 |
| Sodium starch glycolate, NF | 6.0 | 48.0 |
| Silicified microcrystalline cellulose, NF | 1.6 | 13.0 |
| Sodium lauryl sulfate, NF | 0.5 | 4.0 |
| Total | 100.0 | 800.0 |

The composition of the dosage form, the function of the components, and a reference to their quality standards are listed in Table 3.

TABLE 3
Calcium Succinate Formulation by Function

| | | Reference to |
|---|---|---|
| | Function of | Quality |
| Ingredient | Component | Standard* |
| Calcium succinate monohydrate | Active | USP |
| Sodium starch glycolate | Disintegrant | NF |
| Silicified microcrystalline cellulose | Binder | NF |
| Sodium lauryl sulfate | Lubricant | NF |

*In this table and wherever referenced in this document, the terms “NF” and “USP” refer to the National Formulary and U.S. Pharmacopeia, respectively.

### Brief Description of the Manufacturing Process

(A) The manufacturing process for preparation of calcium succinate capsules consisted of the following steps:

| Manufacturing Process | Manufacturing Steps |
|---|---|
| Step 1. Blend | Screen the following pre-weighed formulation ingredients & |
| | transfer to a V-shell blender in the order: |
| | First portion of calcium succinate monohydrate |
| | Silicified microcrystalline cellulose, NF |
| | Second portion of calcium succinate monohydrate |
| | Sodium starch glycolate, NF |
| | Third portion of calcium succinate monohydrate |
| | Blend the ingredients for a time sufficient to provide a first |
| | formulation blend. |
| | Screen sodium lauryl sulfate and transfer to the V-blender. |
| | Blend the ingredients for a time sufficient to provide a |
| | complete formulation blend. |
| Sample for content | Remove in-process samples (A), which will be tested for |
| uniformity | content uniformity. |
| Step 2. Encapsulate | Transfer the complete formulation blend to a Capsule Filling |
| | Machine (the encapsulator). |
| | Add the empty capsules to the encapsulator. |
| | Begin encapsulation. |
| Step 3. Sample for | Periodically remove in-process samples (B), which will be |
| uniformity of dosage form | tested for uniformity of dosage form. |
| Step 4. Package | When encapsulation is complete, package the capsules. |
| Step 5. Sample for batch | Remove samples of the packaged capsules for batch release |
| release | testing. |

(B) The manufacturing process for preparation of a product of the invention consisted of the following steps:

| Manufacturing Process | Manufacturing Steps |
|---|---|
| Step 1. Blend | Screen the following pre-weighed formulation ingredients & |
| | transfer to a V-shell blender in the order: |
| | First portion of calcium succinate monohydrate |
| | Silicified microcrystalline cellulose, NF |
| | Second portion of calcium succinate monohydrate |
| | Sodium starch glycolate, NF |
| | Third portion of calcium succinate monohydrate |
| | Blend the ingredients for a time sufficient to provide a |
| | first formulation blend. |
| | Screen sodium lauryl sulfate and transfer to the V-blender. |
| | Blend the ingredients for a time sufficient to provide a |
| | complete formulation blend. |
| Step 2. Sample for content | Remove in-process samples (A), which will be tested for |
| uniformity | content uniformity. |
| Step 3. Fill stick packs | Transfer the complete formulation blend to a Stick Pack |
| | Filling Machine. |
| Step 4. Sample for | Periodically remove in-process samples (B), which will be |
| uniformity of dosage form | tested for uniformity of dosage form |
| Step 5. Package | When filling is complete, package the stick packs in |
| | cartons. |
| Step 6. Sample for batch | Remove samples of the packaged stick packs for batch |
| release | release testing. |

### Brief Summary of Drug Product Batch Release Specifications

A. The release specifications for the calcium succinate capsule drug product are provided below.

TABLE 4
Calcium Succinate Capsule Release Specifications

| Test | Method | Limits |
|---|---|---|
| Appearance | Visual | White capsule; body printed with “735 mg” in |
| | | black and cap printed with “Product Name” in black |
| Identification: | USP <191> | Capsule contents meet requirements for Calcium |
| Calcium | USP <1065> Ion | Retention time of the main peak matches that of |
| | chromatography with | the standard (+/−5%) |
| | conductivity detection | |
| Identification: | USP <1065> Ion | Retention time of the main peak matches that of |
| Succinate | chromatography with | the standard (+/−5%) |
| | conductivity detection | |
| Water Content | USP <731> LOD at | NMT 2.0% |
| | 105° C. | |
| Assay (Calcium) | USP <1065> Ion | 90.0-110.0% |
| | chromatography with | |
| | conductivity detection | |
| Assay (Succinate) | USP <1065> Ion | 90.0-110.0% |
| | chromatography with | |
| | conductivity detection | |
| Content Uniformity | USP <905> | Meets USP requirements |
| (by weight variation) | | |
| Dissolution | | |
| S1, n = 6 | USP <711>, | Q = 80% at 60 minutes |
| S2, n = 6; if S1 | Apparatus II (water) | S1: Each unit is NLT Q + 5% |
| criteria are not met | | S2: Mean of 12 units (S1 + S2) is equal to or |
| S3, n = 12; if S1 & | | greater than Q, and no unit is less than Q-15% |
| S2 criteria are not met | | S3: Mean of 24 units (S1 + S2 + S3) is equal to or |
| | | greater than Q, not more than 2 units are less than |
| | | Q-15%, and no unit is less than Q-25% |
| Microbial | | |
| Enumeration Tests: | | |
| Total Aerobic | USP <61> | NMT 1000 CFU/g |
| Microbial Count | | |
| Total Yeasts & | | NMT 100 CFU/g |
| Molds Count | | |
| Specified | | |
| Microorganisms | | |
| E. coli | USP <62> | Absent |
| S. aureus | | Absent |
| P. aeruginosa | | Absent |
| Salmonella | | Absent |
| species | | |
| Elemental Impurities | | |
| Cadmium | USP <232>, <233> | NMT 0.5 μg/g |
| Lead | | NMT 0.5 μg/g |
| Arsenic | | NMT 1.5 μg/g |
| Mercury | | NMT 3 μg/g |
| Cobalt | | NMT 5 μg/g |
| Vanadium | | NMT 10 μg/g |
| Nickel | | NMT 20 μg/g |

B. The release specifications for the calcium succinate stick pack drug product are provided below.

TABLE 5
Calcium Succinate Stick Pack Release Specifications

| Test | Method | Limits |
|---|---|---|
| Appearance | Visual | Stick pack is intact and exhibits no visible defects |
| | | in dimensions or labeling. The powder contained |
| | | therein is white to off-white and free-flowing. |
| Identification: | USP <191> | Contents meet requirements for Calcium |
| Calcium | USP <1065> Ion | Retention time of the main peak matches that of |
| | chromatography with | the standard (+/−5%) |
| | conductivity detection | |
| Identification: | USP <1065> Ion | Retention time of the main peak matches that of |
| Succinate | chromatography with | the standard (+/−5%) |
| | conductivity detection | |
| Water Content | USP <731> LOD at | NMT 2.0% |
| | 105° C. | |
| Assay (Calcium) | USP <1065> Ion | 90.0-110.0% |
| | chromatography with | |
| | conductivity detection | |
| Assay (Succinate) | USP <1065> Ion | 90.0-110.0% |
| | chromatography with | |
| | conductivity detection | |
| Content Uniformity | USP <905> | Meets USP requirements |
| (by weight variation) | | |
| Dissolution | | |
| S1, n = 6 | USP <711>, | Q = 80% at 30 minutes |
| S2, n = 6; if S1 | Apparatus II (water) | S1: Each unit is NLT Q + 5% |
| criteria are not met | | S2: Mean of 12 units (S1 + S2) is equal to or |
| S3, n = 12; if S1 & | | greater than Q, and no unit is less than Q-15% |
| S2 criteria are not met | | S3: Mean of 24 units (S1 + S2 + S3) is equal to or |
| | | greater than Q, not more than 2 units are less than |
| | | Q-15%, and no unit is less than Q-25% |
| Microbial | | |
| Enumeration Tests: | | |
| Total Aerobic | USP <61> | NMT 1000 CFU/g |
| Microbial Count | | |
| Total Yeasts & | | NMT 100 CFU/g |
| Molds Count | | |
| Specified | | |
| Microorganisms | | |
| E. coli | USP <62> | Absent |
| S. aureus | | Absent |
| P. aeruginosa | | Absent |
| Salmonella | | Absent |
| species | | |
| Elemental Impurities | | |
| Cadmium | USP <232>, <233> | NMT 0.5 μg/g |
| Lead | | NMT 0.5 μg/g |
| Arsenic | | NMT 1.5 μg/g |
| Mercury | | NMT 3 μg/g |
| Cobalt | | NMT 5 μg/g |
| Vanadium | | NMT 10 μg/g |
| Nickel | | NMT 20 μg/g |

### Stability

All of the formulations/prototypes manufactured as described herein have been stored under the following conditions:

Storage Conditions

| | |
|---|---|
| 25° C./40% Relative Humidity (RH) | Long Term conditions |
| 40° C./75% RH | Accelerated conditions |

Stability Specifications for Calcium Succinate Capsules

(Note: Not all tests were performed at all time points. Tests that were performed at all time points are indicated with a “*”.)

TABLE 6
Calcium Succinate Capsules (Drug Product) Stability Specifications

| Test | Method | Limits |
|---|---|---|
| Appearance* | Visual | White capsule; body printed with “735 mg” in |
| | | black and cap printed with “Product Name” in black |
| Identification: | USP <191> | Contents meet requirements for Calcium |
| Calcium* | USP <1065> Ion | Retention time of the main peak matches that of |
| | chromatography with | the standard (+/−5%) |
| | conductivity detection | |
| Water Content | USP <731> LOD at | NMT 2.0% |
| | 105° C. | |
| Assay (Calcium)* | USP <1065> Ion | 90.0-110.0% |
| | chromatography with | |
| | conductivity detection | |
| Dissolution* | | |
| S1, n = 6 | USP <711>, | Q = 80% at 60 minutes |
| S2, n = 6; if S1 | Apparatus II (water) | S1: Each unit is NLT Q + 5% |
| criteria are not met | | S2: Mean of 12 units (S1 + S2) is equal to or |
| S3, n = 12; if S1 & | | greater than Q, and no unit is less than Q-15% |
| S2 criteria are not met | | S3: Mean of 24 units (S1 + S2 + S3) is equal to or |
| | | greater than Q, not more than 2 units are less than |
| | | Q-15%, and no unit is less than Q-25% |
| Microbial | | |
| Enumeration Tests: | | |
| Total Aerobic | USP <61> | NMT 1000 CFU/g |
| Microbial Count | | |
| Total Yeasts & | | NMT 100 CFU/g |
| Molds Count | | |
| Specified | | |
| Microorganisms | | |
| E. coli | USP <62> | Absent |
| S. aureus | | Absent |
| P. aeruginosa | | Absent |
| Salmonella | | Absent |
| species | | |

Calcium Succinate Capsule Stability

Six batches of calcium succinate capsules were manufactured in compliance with Current Good Manufacturing Practices, released after meeting rigorous release specifications (Table 4, above), and placed in stability storage under the conditions described above. Equivalence between the six batches of calcium succinate capsules has been confirmed by comparison of stability data.

Appearance

The appearance of the calcium succinate capsules did not change during storage at 25° C./60% relative humidity (RH) or 40° C./75% RH.

Assay (Percent Calcium)

At each of nine time points (T=0, 1, 2, 3, 6, 9, 12, 18 and 24 months), the percent calcium in calcium succinate capsules was determined by ion chromatography. Data are summarized in Table 7.

TABLE 7
Assay (Percent Calcium) in Calcium Succinate
Capsules after Storage

| | Batch Number | | | | | |
|---|---|---|---|---|---|---|
| | 1 | 2 | 3 | 4 | 5 | 6 |
| Months of Storage at | | | | | | |
| 25° C./40% RH | | | | | | |
| 0 | 92.7 | 93.5 | 100.4 | 100.0 | 100.2 | 99.9 |
| 1 | 93.0 | 95.0 | 100.1 | 100.4 | 99.8 | 99.5 |
| 2 | 93.8 | 95.7 | 103.4 | 100.5 | 100.9 | 100.8 |
| 3 | 95.7 | 96.8 | 107.0 | 99.7 | 99.7 | 99.6 |
| 6 | 97.9 | 97.6 | 102.6 | 100.0 | 99.9 | 100.2 |
| 9 | 97.2 | 96.5 | 102.3 | 100.2 | 98.9 | 99.4 |
| 12 | 99.2 | 94.4 | 101.7 | 99.9 | 101.2 | 100.9 |
| 18 | 95.9 | 94.2 | 103.6 | 99.5 | 101.3 | 101.3 |
| 24 | 98.0 | 95.1 | 97.3 | 98.9 | 99.8 | 99.1 |
| Months of Storage at | | | | | | |
| 40° C./75% RH | | | | | | |
| 0 | 92.7 | 93.5 | 100.4 | 100.0 | 100.2 | 99.9 |
| 1 | 91.3 | 95.4 | 97.8 | 100.2 | 100.0 | 100.5 |
| 2 | 94.8 | 95.7 | 101.0 | 100.5 | 100.3 | 100.6 |
| 3 | 101.5 | 100.1 | 104.1 | 100.3 | 101.6 | 102.2 |
| 6 | 98.3 | 95.4 | 103.7 | 100.5 | 100.2 | 100.3 |
| 9 | 95.8 | 93.7 | 101.0 | 98.0 | 99.1 | 98.8 |
| 12 | 99.3 | 98.1 | 104.2 | 99.8 | 97.8 | 98.8 |

Dissolution

At each of nine time points (Time 0, and 1, 2, 3, 6, 9, 12, 18 and 24 months), dissolution was performed in accordance with USP <711> Dissolution, Apparatus II (paddles) and the dissolution medium water, using a qualified and calibrated dissolution system. A validated analytical method for the determination of calcium was used to monitor calcium release from a capsule. The extent of calcium release during dissolution was determined.

TABLE 8
Percent Calcium Release by Dissolution of Capsules after Storage

| | Batch Number | | | | | |
|---|---|---|---|---|---|---|
| | 1 | 2 | 3 | 4 | 5 | 6 |
| Months of Storage at | | | | | | |
| 25° C./40% RH | | | | | | |
| 0 | 91 | 90 | 92 | 94 | 96 | 94 |
| 1 | 86 | 86 | 96 | 95 | 95 | 99 |
| 2 | 90 | 87 | 94 | 96 | 94 | 99 |
| 3 | 89 | 86 | 94 | 95 | 96 | 96 |
| 6 | 89 | 98 | 96 | 94 | 92 | 96 |
| 9 | 88 | 89 | 92 | 92 | 88 | 93 |
| 12 | 92 | 90 | 91 | 88 | 86 | 92 |
| 18 | 93 | 92 | 92 | 91 | 92 | 100 |
| 24 | 90 | 91 | 100 | 89 | 93 | 86 |
| Months of Storage at | | | | | | |
| 40° C./75% RH | | | | | | |
| 0 | 91 | 90 | 92 | 94 | 96 | 94 |
| 1 | 89 | 87 | 91 | 97 | 93 | 97 |
| 2 | 90 | 88 | 99 | 95 | 99 | 100 |
| 3 | 90 | 86 | 95 | 95 | 97 | 95 |
| 6 | 98 | 95 | 92 | 97 | 96 | 95 |
| 9 | 90 | 88 | 93 | 92 | 91 | 87 |
| 12 | 86 | 87 | 93 | 88 | 87 | 87 |

Microbial Limits

All batches of calcium succinate capsules passed microbial limits tests during storage at 25° C./60% relative humidity (MI).

### Brief Description of the Dissolution Assay

Under the validated conditions, dissolution was performed in accordance with USP <711> Dissolution, using Apparatus II (paddles) rotated at 50 RPM and 900 mL of the dissolution medium water at 37±0.5° C. The dissolution system was qualified and calibrated. An aliquot of the fluid in a dissolution vessel was removed at 5, 10, 15, 20, 30, 45, and 60 minutes after initial test sample exposure. The percent of calcium released relative to the label claim was determined, correcting for the volume change in the vessel associated with sample withdrawal. The in vitro dissolution profiles encompassed the timeframe over which at least 85% of the drug was dissolved.

If a calcium succinate capsule was tested, the capsule was weighed before testing and the weight was recorded. A Teflon-coated sinker (Quality Lab Accessories, 6880 Green Valley Circle, Aubrey, TX 76227; Part No. CAPWHT-XL) that was appropriately sized to contain a size 00 capsule was attached to each capsule to prevent the capsule from floating after exposure to the dissolution medium.

If a calcium succinate stick pack was tested, the contents of a stick pack were quantitatively transferred to a dissolution vessel for testing as described above.

### Brief Description of the Problem

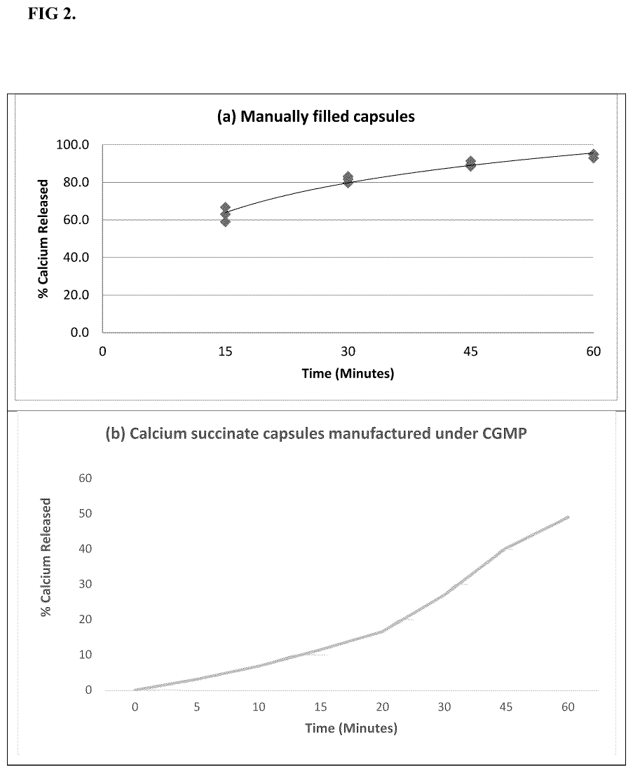

A batch (Batch Number 7) was manufactured in compliance with Current Good Manufacturing Practices. Samples of the calcium succinate capsules drug product manufactured as part of Batch Number 7 were submitted for release testing. The capsules from this batch met all release requirements except the requirements for dissolution. Encapsulated Drug Product in this batch failed to dissolve and release at least 90% of the calcium ion within 60 minutes. (See FIG. 2b.) The batch was not released or placed in stability storage.

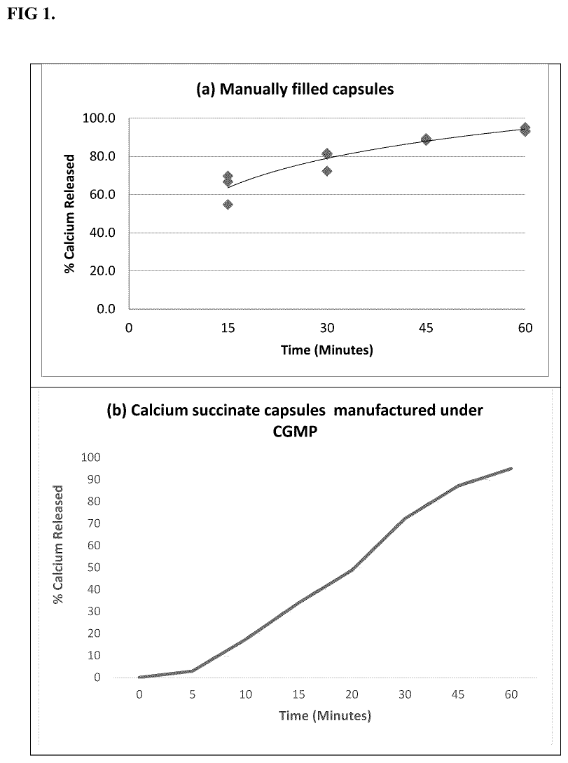

In order for a Drug Product to be identified as an “Immediate Release” solid oral drug product containing a high solubility drug substance, the dissolution criterion is Q=80% in 30 minutes. The data in FIGS. 1(b) and 2(b) show that calcium succinate capsules that are manufactured at commercial scales may not meet this criterion. Therefore, a root cause analysis was carried out and additional experiments were performed to discover the reasons for failure to meet the dissolution requirement set by the Release Specifications and unique formulations that might qualify as “Immediate Release” solid oral drug products.

### Root Cause Analysis

The unexpected test results associated with Batch Number 7 prompted the re-examination and re-evaluation of the impacts of changes to the raw materials, manufacturing site, manufacturing scale, manufacturing equipment, manufacturing personnel, dissolution test sites, dissolution procedure, dissolution test system, and analysts. This evaluation confirmed that the site, personnel and equipment were appropriate and qualified for manufacturing and testing calcium succinate capsules Drug Product. No changes were made to the manufacturing personnel, dissolution test sites, dissolution procedure, dissolution test system, and analysts, eliminating these as potential causes of the failure of Batch Number 7 to meet release specifications for dissolution.

A review of the published literature indicated the following:
Tamp filling encapsulators typically consist of a powder bowl situated over a dosing disk and a series of tamping stations. Powder flows into the holes of the dosing disk during indexing between tamping stations and a powder plug is formed by successively compacting the powder at each tamping station. The penetration depth of the tamping pin into the dosing disk controls the compacting pressure, which typically ranges from 50 to 150 Newtons. To improve capsule weight uniformity, the tamping settings are adjusted to provide a constant and reproducible densification of the powder formulation into a plug. At the final station, the plug is ejected into the body of the capsule, the cap replaced and the capsule closed. The settings of the encapsulating machine are typically adjusted to ensure consistent and reproducible filling of the capsule to the target weight.
Shah et al. have reported that both the number of tamps and the tamping force, as well as formulation variables, could influence the drug release rate from an encapsulated dosage form. [See: K B Shah, et al. Multiple tamping effects on drug dissolution from capsules filled on a dosing-disk type automatic capsule filling machine. Journal of Pharmaceutical Sciences, Vol. 76, No. 8, pages 639-645, August 1987.] Generally, Shah et al. observed a trend toward slower dissolution rate with increasing numbers of tamps, the effect being most marked when calcium phosphate dihydrate (a calcium salt) was the filler. When compression was measured, high compression adversely affected drug release from the dicalcium phosphate-based capsules that were studied.
As Hardy et al. acknowledge: “The literature on capsule technology is relatively sparse, with those pertaining to tamp filling processes at a premium.” [See: I J Hardy, et al. Rational design of powder formulations for tamp filling processes. Journal of Pharmacy and Pharmacology, Vol. 55, pages 1593-1599, 2003.] If drug dissolution is slow, Hardy et al. recommend reducing tamping force as a potential corrective action. However, Hardy also cautions that reducing tamping force may result in a weaker plug, reducing the uniformity of capsule filling.

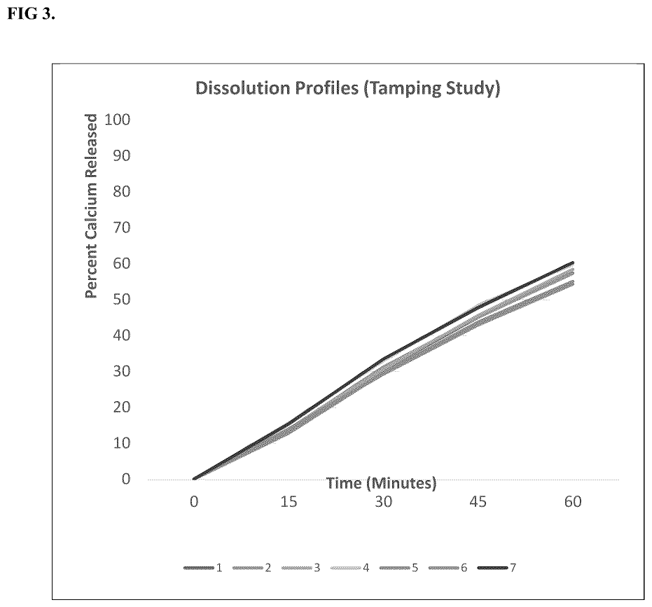

Research Results
Manually filled capsules—Experiments were carried out in which the dissolution profiles of capsules that were manually filled with a formulation containing a specific lot of calcium succinate were compared to the dissolution profiles of Drug Product capsules containing the same lot of calcium succinate that were manufactured at commercial scale. The data indicated that compaction during the commercial-scale encapsulation process caused calcium release from the capsules to be slower and less extensive (FIGS. 1a and 1b and 2a and 2b). Note that the dissolution profile shown in FIG. 2b fails to meet the current dissolution specification for Drug Product.
The data also indicated that if the compaction pressures associated with tamp filling could be reduced by adjustment of equipment parts and/or operating parameters, an acceptable dissolution profile for the calcium succinate capsule may be obtained.
Tamping Study of Batch 8 Capsules—Taken together, the data presented in FIGS. 1 and 2 indicated that commercial-scale encapsulation must take place with less compaction (i.e., less tamping pressure) during encapsulation to ensure rapid and extensive release of calcium from calcium succinate Drug Product capsules. Therefore, completion of a tamping study was authorized in which the tamping springs on the encapsulating machinery at the commercial manufacturing site were adjusted in six different ways in attempts to reduce compaction of the blend while maintaining the “plug” formation required for the machine to transfer the material into the capsule body. The data (FIG. 3) showed that all attempts to alter tamping spring settings failed to reduce the formulation compaction that took place during commercial-scale encapsulation and failed to prevent its adverse effects on the dissolution profile.
Preparation of Formulation in Stick Packs—The calcium succinate formulation described in Table 2 was prepared and transferred as approximately 800 mg portions of the formulation to empty stick packs. The filled stick packs were closed and sealed. Stick pack units were placed on stability. From time to time, units were removed from stability storage and dissolution of the powder was tested as described above. All units released at least about 80% of the calcium contained in the formulation within 15 minutes at all time points.
Brief Summary of Discovery—Based on the research described above, it has been discovered that a calcium succinate formulation as disclosed herein exhibits chemical and physical stability for 1 year when stored at 40° C./75% RH and for at least 3 years when stored at 25° C./40% RH. The formulation has no undesirable taste or odor (i.e., the formulation is palatable). The formulation does not degrade chemically or physically. Further, if the formulation is subjected to dissolution testing, the powder consistently releases at least about 80% of the calcium contained in the formulation within about 30 minutes. Thus, the present invention provides a unique, ready to use, orally administered formulation containing a therapeutically effective amount of calcium as calcium succinate monohydrate with unexpected palatability, chemical and physical stability, and availability for phosphate binding in the gastrointestinal tract of a subject, wherein the formulation is provided in a ready to use stick pack. A formulation of her invention meets the requirement for an “Immediate Release” Drug Product.

The invention claimed is:

1. A calcium succinate formulation in a ready to use stick pack, the formulation comprising:
a therapeutically effective amount of calcium succinate,
from about 5.5 to about 6.5 weight percent of a disintegrant,
from about 1.4 to about 1.6 weight percent of a binder,
from about 0.4 to about 0.6 weight percent of a lubricant, wherein said formulation releases at least about 80% of the calcium within 30 minutes when dissolved in water at a temperature of 37±0.5° C. that is stirred by paddles rotating at a rate of 50 RPM.

2. The calcium succinate formulation in a ready to use stick pack of claim 1, wherein the disintegrant is sodium starch glycolate.

3. The calcium succinate formulation in a ready to use stick pack of claim 1, wherein the binder is silicified microcrystalline cellulose.

4. The calcium succinate formulation in a ready to use stick pack of claim 1, wherein the stick pack comprises a laminate sheet that is in contact with the formulation and wherein the laminate sheet is formed from polyethylene or polypropylene.

5. The calcium succinate formulation in a ready to use stick pack of claim 1, wherein the stick contains a single dosage of calcium succinate.

6. The calcium succinate formulation in a ready to use stick pack of claim 5, wherein the single dosage of calcium succinate is from about 109 to 209 mg calcium succinate.

7. The calcium succinate formulation in a ready to use stick pack of claim 1, wherein the stick pack contains a double dosage of calcium succinate.

8. The calcium succinate formulation in a ready to use stick pack of claim 7, wherein the double dosage of calcium succinate is from about 188 to 388 mg calcium succinate.

9. The calcium succinate formulation in a ready to use stick pack of claim 1, wherein the stick pack contains a triple dosage of calcium succinate.

10. A method of treating hyperphosphatemia in a patient in need thereof comprising administering to said patient a therapeutically effective amount of the formulation of claim 1.
